(19) United States
(12) Patent Application Publication
(10) Pub. No.: US 2021/0401876 A1
(43) Pub. Date: Dec. 30, 2021

(54) PHARMACEUTICAL COMPOSITION OF CHLORINE FOR TREATMENT OF RESPIRATORY VIRAL INFECTION

(71) Applicant: Lifeactivus Private Limited, Hyderabad (IN)

(72) Inventors: Keshav DEO, Hyderabad (IN); Dinesh R. PANCHASARA, Gujarat (IN)

(21) Appl. No.: 17/275,086

(22) PCT Filed: Oct. 15, 2020
(86) PCT No.: PCT/IB2020/059672

§ 371 Date: Mar. 10, 2021

(30) Foreign Application Priority Data
Apr. 21, 2020 (IN) 202041017016

(51) Int. Cl. A61K 33/00; A61K 47/02; A61K 9/00; A61P 31/14

(52) U.S. Cl. CPC A61K 33/00; A61P 31/14; A61K 9/0073; A61K 47/02

(57) ABSTRACT

The present invention relates to a pharmaceutical composition of chlorine for treatment of respiratory viral infection. More particularly, the invention relates to the pharmaceutical composition of chlorine administered by inhalation for the treatment of viral infection associated with COVID and viral morbidities.

### RELATED APPLICATIONS

The present application claims the benefit of priority to Indian provisional patent application No 202041017016 filed on Apr. 21, 2020 and entire provisional specification.

### FIELD OF THE INVENTION

The present invention relates to a pharmaceutical composition of chlorine for treatment of respiratory viral infection. More particularly, the invention relates to the pharmaceutical composition of chlorine administered by inhalation for the treatment of respiratory viral infection. The present invention also provides a method for the management of respiratory viral infection associated with COVID and viral morbidities comprising administering pharmaceutically effective amount of chlorine by inhalation.

### BACKGROUND OF THE INVENTION

The virus, technically named SARS-CoV-2, is a newly identified virus, but it is the seventh Coronavirus known to infect humans. The resulting illness is referred to as COVID-19. This virus is in the same Coronavirus family as severe acute respiratory syndrome Coronavirus (SARS-CoV or SARS) and Middle East respiratory syndrome Coronavirus (MERS-CoV or MERS), which caused the two previous Coronavirus outbreaks. Since SARS and MERS are from the same family of coronaviruses, they have similar physical and biochemical properties and comparable transmission routes as COVID-19. In the absence of COVID-19 specific data, we rely on SARS, MERS, and coronavirus surrogate data to extrapolate, assess, and manage risk. COVID-19 is the infectious disease caused by the most recently discovered coronavirus. This new virus and disease were unknown before the outbreak began in Wuhan, China, in December 2019.

The virus causes mild to moderate symptoms. When the infection is limited to upper respiratory tract, fever, tiredness, Shortness of breath and dry cough are symptoms; but when the infection involves the lower respiratory tract, the virus damages lungs which leads to shortness of breath. The oxygen transfer capacity of the organs gets affected. Thus oxygen level in the blood decreases which affects brain, heart and then other organs. When patients already have serious or chronic conditions, it is possible that the viral infection can cause septic shock or multi organ dysfunction or failure.

Scientists are trying hard to find drugs to treat COVID-19. According to research there are more than 30 agents including Western medicines, natural products and traditional Chinese medicines that may have potential efficacy against COVID-19. However, a number of medicines have been suggested as potential investigational therapies, many of which are now being or will soon be studied in clinical trials, including the SOLIDARITY trial co-sponsored by WHO and participating countries. Several drugs such as chloroquine, arbidol, remdesivir, and favipiravir are currently undergoing clinical studies to test their efficacy and safety in the treatment of coronavirus disease 2019 (covid-19).

Favipiravir, an antiviral that has shown potential in treating the novel coronavirus and is approved for marketing in the treatment of influenza, and is one of three drugs demonstrating efficacy against the novel coronavirus in human trials. Zhejiang Hisun Pharmaceutical Company, has met requirements to produce the generic form of Favipiravir. Human trials conducted in Shenzhen, Guangdong province, the drug has shown promising results against the novel coronavirus and mild adverse reactions in patients. Controlled Study of the Efficacy of Lopinavir Plus Ritonavir and Arbidol for treating with patients with novel coronavirus infection is in Phase-4.

Viruses are divided into three groups: enveloped viruses are surrounded by an outer lipid membrane; non-enveloped viruses (divided into large non-enveloped and small non-enveloped) lack this membrane. Where present, the envelope contains the viral proteins, which mediate binding to host cells. Viral genetic material is packaged inside protein structures called capsids. Crucially, enveloped viruses are easier to kill: SARS-CoV-2, the virus responsible for the COVID-19 outbreak, is an enveloped virus and therefore the easiest to kill.

It is discovered that each disinfectant has totally different effects, attacking one or several of the virus' functions. Even though the outcome is the same, the eradication methods are different. Ozone, ultraviolet irradiation, liquid chlorine, chlorine dioxide, and sodium hypochlorite disinfections are commonly used technologies for disinfection. Using the correct disinfectant is an important part of preventing and reducing the spread of illnesses. Isopropanol or Ethanol (Alcohol), Quaternary Ammonium Compounds, Sodium Hypochlorite (Bleach), Hydrogen Peroxide are some common disinfectants which kills viruses, bacteria as well as fungi. Chlorine is a kind of strong oxidizer, which is one of the most early used disinfection methods. WHO recommended use of chlorine to make inactive COVID-19 for treatment of drinking water and swimming pools.

U.S. Pat. No. 9,782,434 discloses method for treating or preventing infection which results from viruses, bacteria, and fungi. Method provides administering oxidative reductive potential water solution comprises free chlorine species at a level of about 10 ppm to about 400 ppm, as liquid, steam, aerosol, mist or spray. But using this formulation for inhalation purpose the level of chlorine in the solution is too high according to the AEGL values describe the expected effects of inhalation exposure to certain compounds including Chlorine. U.S. Pat. No. 6,333,054 discloses topic hydrogel disinfectant composition comprising active chlorine.

Various non-patent literatures disclosed the Effects of a Low Concentration hypochlorous acid (HOCl). Hyun Jik Kim et al (Laryngoscope 118: October 2008) disclose Effects of a Low Concentration hypochlorous acid nasal irrigation solution on bacteria, fungi, and viruses. This literature disclosed the virucidal effects of HOCl and used the human influenza a virus to challenge the cells. TaylanGun et al (J Ann Eu Med 2018; 6(3): 37-9) disclose efficacy of low-concentration hypochlorous acid spray in acute sore throat relief.

Inhalation therapy is the best option for respiratory diseases. Thus, the present invention discloses pharmaceutical composition of chlorine by inhalation for treatment of respiratory viral infection.

The treatment for COVID-19 is symptomatic because there is no specific drug or cure for it. It is essential to develop effective, cheap, user friendly method of treating COVID-19.

Thus, the inventors of the present invention have successfully addressed the existing drawbacks and formulated pharmaceutical composition for treatment of viral respiratory infection. The inventors of the present invention have successfully formulated the pharmaceutical composition of chlorine administered by inhalation for the treatment of respiratory viral infection.

### Objects

An object of the present disclosure is to develop effective, inexpensive and user friendly method of treating respiratory viral infection.

An object of the present disclosure is to provide the pharmaceutical composition of chlorine for treatment of respiratory viral infection.

An object of the present disclosure is to provide the pharmaceutical composition of chlorine by inhalation for treatment of respiratory viral infection.

Another object of the present disclosure is to provide method of treatment of respiratory viral infection comprises inhalation of a therapeutically effective amount of chlorine.

Yet another object of the present disclosure is to provide the pharmaceutical composition comprising aqueous solution of chlorine to treat COVID-19.

### SUMMARY OF THE INVENTION

The present disclosure envisages a liquid composition of chlorine for treatment of respiratory viral infection.

In one aspect the present invention provides the pharmaceutical composition administered by inhalation in an individual; wherein the concentration of chlorine is in the range of 0.1 to 2 ppm on the basis of breathing air for 15 min.

In one embodiment the present invention provides the composition; wherein inhalation time is 15 min at interval of 4 hrs for 3 days.

In another embodiment the present invention provides the composition; wherein the concentration of chlorine by inhalation is in the range of 0.1 to 1 ppm on the basis of breathing air for 15 min at interval of 2 hrs for 6 days.

In another embodiment the present invention provides the composition; wherein the chlorine inhalation by Individual is in the range of 0.1 to 2 ppm on the basis of breathing air for 5 min at interval of 2 hrs for 6 days.

Yet, in another embodiment the present invention provides the composition; wherein the chlorine inhalation by Individual is in the range of 0.1 to 2 ppm on the basis of breathing air for 10 min at interval of 6 hrs for 6 days.

In another embodiment the present invention provides the composition; wherein the chlorine inhalation by Individual is in the range of 0.1 to 2 ppm on the basis of breathing air for 10 min at interval of 12 hrs for 12 days.

In another aspect the present invention provides the pharmaceutical composition administered by inhalation from 1 to 10 times per day.

In another aspect the present invention provides the pharmaceutical composition comprising an effective amount of chlorine dissolved in an inactive ingredient and/or a stabilizing agent.

In another aspect the present invention provides the pharmaceutical composition; wherein the chlorine content is in the range of 1 to 200 ppm.

In another embodiment the present invention provides the composition; wherein the chlorine content is in the range of 1 to 99 ppm.

In another embodiment the present invention provides the composition; wherein said chlorine is chlorine gas or chlorine releasing compound.

In another embodiment, wherein chlorine releasing compounds are selected from mixture of calcium hypochlorite with the salt of sodium, potassium, ammonia; wherein salt of sodium, potassium, ammonia are sodium sulphate, potassium sulphate and ammonium sulphate.

Yet in an another embodiments, wherein said inactive ingredient is selected from distilled water, saturated edible oil, saturated neuzoil oil, saline water, glycols, higher alcohol and mixture thereof. More particularly, said inactive ingredient is distilled water.

In another embodiment the present invention provides the composition; wherein said stabilizing agents is selected from alkali salt of mineral acids, saline water, sodium sulphate, sodium carbonate, sodium bicarbonate, potassium sulphate, potassium chloride, boric acid and mixture thereof. The concentration of said stabilizing agent is in the range of 0.1 to 10%.

In another embodiment the present invention provides the composition; wherein pH of solution is in the range of 1 to 8.

In accordance with another aspect the invention provides a process of preparing aqueous chlorine solution for inhalation, comprising the steps of:

(a) Dissolving chlorine into inactive ingredient;

(b) Adding stabilizing agent to solution of step (a);

(b) Sterilizing by filtration method and

(c) Filling in pre-sterilized vials or added into respule and sealed with closure.

According to another embodiment, provides a process of preparing aqueous chlorine solution for inhalation, wherein the sterilization of step (b) is carried out through 0.22μ filter. According to another embodiment, provides a process of preparing aqueous chlorine solution for inhalation, wherein pre-sterilized vials comprising aqueous chlorine solution, aseptically packed in amber colour glass vial of 10 mL and each consisting of 100 ppm concentration of chloride.

In accordance with another aspect the invention provides a method of treating a respiratory viral infection which includes administrating aqueous chlorine solution described hereinabove by inhalation.

According to another embodiment, the invention provides a method of treatment of respiratory viral infection which includes administrating aqueous chlorine solution described hereinabove by inhalation using nebulizer.

While several embodiments have been described in detail, it will be apparent to those skilled in the art that the disclosed embodiments may be modified. Therefore, the foregoing description is to be considered exemplary rather than limiting.

### BRIEF DESCRIPTION OF THE DRAWING

The following figures are illustrative of particular examples for enabling embodiments of devices and methods of the present disclosure, are descriptive of some of the embodiments and are not intended to limit the scope of the disclosure. The figures are not to scale (unless so stated) and are intended for use in conjunction with the explanations in the following detailed description. Wherever applicable, the words and phrases used herein should be understood and interpreted to have a meaning consistent with the understanding of those words and phrases by those skilled in the relevant art. Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

FIG. 1 illustrates effects of five common disinfectants on bacteriophage MS2.

FIG. 2 illustrates disinfection (2−log) of microorganisms by free available chlorine.

FIG. 3 illustrates combination of chlorination and ultra-violate light against inactivation of viruses.

FIG. 4 illustrates solubility of chlorine gas.

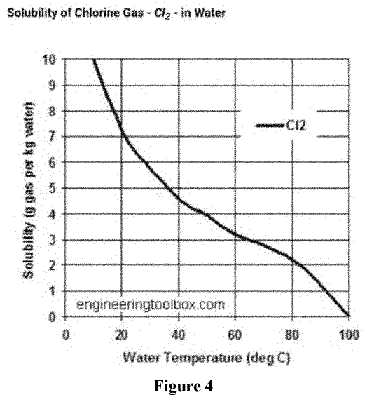

FIG. 5 illustrates mechanism of action of liquid composition disclosed in the present invention.

FIG. 6: illustrates the (A) LifeViroTreat unit vial, (B) method of application by nebulizer and (C) LifeViroTreat pilot batch for clinical testing.

### DESCRIPTION

In the description that follows, a number of terms are used, the following definitions are provided to facilitate understanding of various aspects of the disclosure. Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

The terms and words used in the following description are not limited to the bibliographical meanings, but, are merely used to enable a clear and consistent understanding of the disclosure. Accordingly, it should be apparent to those skilled in the art that the following description of exemplary embodiments of the present disclosure are provided for illustration purpose only and not for the purpose of limiting the disclosure as defined by the appended claims and their equivalents.

Features that are described and/or illustrated with respect to one embodiment may be used in the same way or in a similar way in one or more other embodiments and/or in combination with or instead of the features of the other embodiments.

The therapeutically effective amount administered to the patient, e.g., a mammal, particularly a human, in the context of the present invention should be sufficient to affect a therapeutic or prophylactic response in the patient over a reasonable time frame. The dose can be readily determined using methods that are well known in the art. One skilled in the art will recognize that the specific dosage level for any particular patient will depend upon a variety of potentially therapeutically relevant factors.

The term “subject” includes mammals (especially humans) and other animals, such as domestic animals (e.g., household pets including cats and dogs) and non-domestic animals (such as wildlife).

Viral infections may include for example infections by respiratory viruses, including but not limited to, various types of influenza, such as influenza A, influenza B and numerous other strains of influenza, including seasonal, avian (e.g., H5N1 strains), COVID, and swine (e.g., H1N1 strains).

“Breathing air” is a term used to loosely describe the extent to which air in a local environment (ambient air) is sufficiently to be safe to breathe (Rajagopal Kannan et al, IJISET, Vol. 2 Issue 4, April 2015).

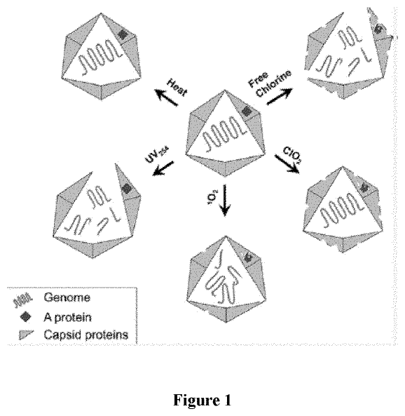

Wigginton et. al discloses effects of five common disinfectants (free chlorine (FC), singlet oxygen (O₂), chlorine dioxide (ClO₂), UV radiation, and heat) on bacteriophage MS2, an icosahedral virus with a single stranded RNA genome (3690 nt) that encodes four proteins (FIG. 1). Poliovirus inactivation by FC has been independently attributed to both protein damage and RNA damage. In terms of specific function losses, FC inactivation of adenovirus was attributed to damage in viral proteins necessary for genome delivery, whereas FC treatment of human picornaviruses and feline caliciviruses was attributed to a loss in host cell recognition.

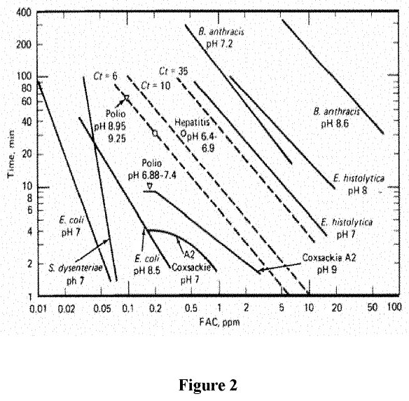

According to the study by Baker et al. theorized that chlorine can destroys microorganisms by combining with proteins to form N-chloro compounds. Temperature, over the range appropriate for drinking-water, affects the rate of disinfection reactions according to the Arrhenius equation, although this may not hold for certain disinfectants at low temperatures. The pH of the disinfectant solution affects the reaction kinetics. Moreover, the disinfection efficiency of free chlorine is increased at lower pH values, whereas that of chlorine dioxide is greater at alkaline pH levels. Disinfection (2−log) of microorganisms by free available chlorine depicted in FIG. 2.

According to White's handbook of chlorination and alternative disinfectants: Wiley; 2010 Chlorination effectively inactivates viruses if the turbidity of the water is less than or equal to 1.0 nephelometric turbidity unit. It requires free chlorine residual of 1.0 or greater for 30 minutes, and a pH of less than 8.0. White in 2010 in his book studied about chlorination and its ability as a disinfectant and concludes in a relative chart between time and concentration of chlorine.

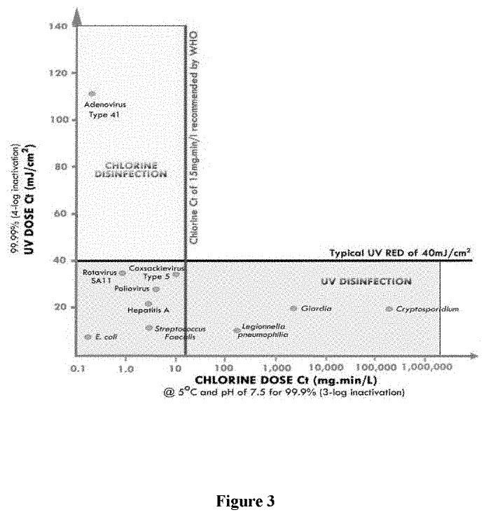

According to Environmental Health Services, U.S. Department of Health & Human Services advisory and guidelines exposure to 0.5 mg/l free chlorine for at least 30 minutes is one of the best measures to kill virus in drinking water. Environmental Health Services also advice on COVID-19 that a predetermined relative efficacy of chlorination and ultraviolet light is effective in the inactivation of various viruses in drinking water (FIG. 3).

Chlorine group comprises aqueous solution of chlorine, hypochlorite, or hypochlorous acid. Occasionally, chlorine-releasing compounds and their salts are included in this group. Frequently, a concentration of <1 ppm of available chlorine is sufficient to kill bacteria and viruses, spores.

In humans, at 1-3 ppm, there is mild mucus membrane irritation that can usually be tolerated for about an hour. At 5-15 ppm, there is moderate mucus membrane irritation. At 30 ppm and beyond, there is immediate substernal chest pain, shortness of breath, and cough. Acute Exposure Guideline Levels (AEGLs) set levels of chemical concentration that pose a defined level of risk to humans. The AEGL values are determined for varying times of exposure, such as ten minutes, thirty minutes, one hour, four hours and eight hours.

The AEGL values describe the expected effects of inhalation exposure to certain compounds (airborne concentrations in ppm or mg/m3). Each AEGL is determined by different levels of a compound's toxicological effects, based on the 4 Ds: detection, discomfort, disability and death.

There are three levels of AEGL-values: AEGL-1, AEGL-2 and AEGL-3. AEGL-1 is the airborne concentration above which notable discomfort or irritation could be experienced. However, the effects are not disabling and reversible once exposure stops. AEGL-2 is the airborne concentration above which irreversible or other serious, long-lasting adverse health effects or an impaired ability to escape could be experienced. AEGL-3 is the airborne concentration above which life-threatening health effects or death could be experienced.

TABLE 1
Summary and Relationship of AEGL Values for chlorine
gas (Extracted from NIH: Ref. Acute Exposure Guideline
Levels for Selected Airborne Chemicals: Volume 4)

| | Exposure Duration | | | | |
|---|---|---|---|---|---|
| Classification | 10 min | 30 min | 1 h | 4 h | 8 h |
| AEGL-2 | 2.8 ppm | 2.8 ppm | 2.0 ppm | 1.0 ppm | 0.71 ppm |
| (Disabling) | (8.1 mg/m³) | (8.1 mg/m³) | (5.8 mg/m³) | (2.9 mg/m³) | (2.0 mg/m³) |
| AEGL-3 | 50 ppm | 28 ppm | 20 ppm | 10 ppm | 7.1 ppm |
| (Lethal) | (145 mg/m³) | (81 mg/m³) | (58 mg/m³) | (29 mg/m³) | (21 mg/m³) |

Abbreviations: mg/m3, milligrams per cubic meter; ppm, parts per million.

Thus, the present disclosure envisages a pharmaceutical composition of chlorine for treatment of respiratory viral infection. More particularly provides a pharmaceutical composition comprising of chlorine dissolved in inactive ingredient; optionally adding stabilizing agent.

In an embodiment the present invention disclosed a composition comprises the chlorine gas is dissolved in the WFI grade water to form a chlorine gas solution at 0-10° C. if desired, can be combined with one or more suitable carriers, e.g., vehicles, adjuvants, excipients, diluents, and the like.

In an embodiment the present invention disclosed the composition comprises the chlorine gas is dissolved in the WFI grade water to form a chlorine gas solution at 0-10° C.

In an embodiment the present invention disclosed the composition; wherein chlorine content is in the range of 1 to 200 ppm. More particularly in the range of 1 to 99 ppm.

In an embodiment the present invention disclosed method of administering the composition by inhalation; wherein the concentration of chlorine is in the range of 0.1 to 2 ppm on the basis of breathing air for 15 min.

In an embodiment the present invention disclosed a composition stored in an amber color glass vial.

Mechanism of Action

Not all chlorine species are equally effective as disinfectants. Many studies have explored the mechanism of chlorine disinfection.

Inactivation occurs by means of one or more of the following mechanisms:

1. Inactivation of the key enzymes

2. Disruption of nucleic acids rendering them non-functional

3. Oxidative damage to cell walls or other vital cell components

Effectiveness of each disinfecting agent is a function of both its rate of diffusion through the cell wall and its reactivity with the cell wall, proteins and nucleic acid.

Viruses associated with cellular debris or organic particles may require high levels of disinfection due to the protective nature of the particle surface. Viral genetic material is packaged inside protein structures. Enveloped viruses are surrounded by outer lipid membrane. SARS-CoV-2 is an enveloped virus and therefore the easiest to kill.

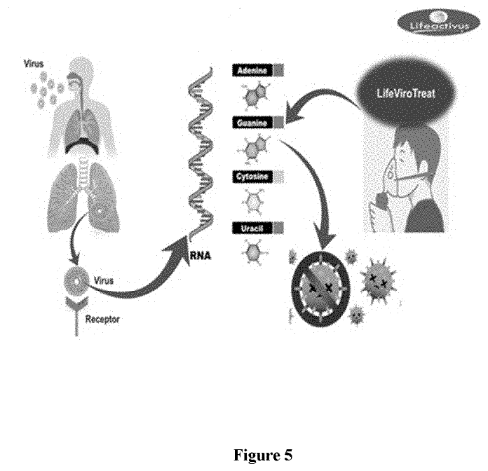

The mechanism of action of liquid composition disclosed in the present invention depicted in FIG. 5 which indicates that the liquid composition of present invention administered by inhalation kill the viruses in respiratory tract. The chlorine solution of present invention reacts with RNS protein and kills viruses.

Nebulizer for Inhalation of Aqueous Chlorine:

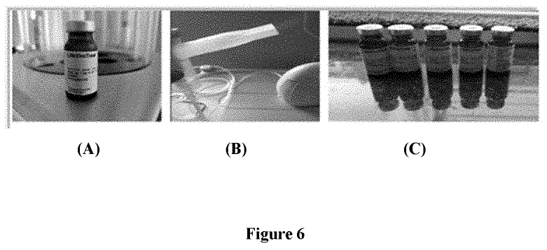

A nebulizer breathing treatment may be an effective way to ensure quick and thorough relief for patients with some respiratory ailments. A nebulizer is a machine that helps you to breathe in a medicine as a mist through a mask or a mouthpiece. Pressurized air passes through the tube and turns the liquid medicine into a mist. During an asthma attack or a respiratory infection, the mist may be easier to inhale than the spray from a pocket inhaler. When airways become narrow one cannot take deep breaths, it provides immediate relief by the opening of airways. For this reason, a nebulizer is a more effective way to deliver the medication. They come in electric or battery-run versions. They come in both a portable size you can carry with you and a larger size that's meant to sit on a table and plug into a wall. Both are made up of a base that holds an air compressor, a small container for liquid medicine, and a tube that connects the air compressor to the medicine container. Above the medicine container is a mouthpiece or mask you use to inhale the mist (FIG. 6 B).

A Respule is a small plastic/glass container that contains a liquid in a dosage form. The liquid is put into a machine called a nebulizer. Before using nebulizer wash the hands, and then adding the vial/respule to the medicine cup. Assemble the top piece, tubing, mask, and mouthpiece. Attach the tubing to the machine, according to the instructions. Turn the nebulizer on. While using the nebulizer, hold the mouthpiece and medicine cup upright to help deliver all the medication. Take slow, deep breaths through the mouthpiece and inhale all the medicine.

The present invention can be administered in any suitable form in accordance with the present invention, e.g., as spray, mist, aerosol or steam.

The present invention is used for treating or preventing respiratory viral infection associated with an upper respiratory condition. When the infection is associated with an upper respiratory condition or lower respiratory tract infection including lung tissue, the composition of present invention is preferably administered to the upper airway, e.g., as a spray, mist, aerosol or steam, so as to contact one or more upper airway tissues, lower respiratory tract infection including lung tissue affected by the condition. Any suitable method can be employed for delivering the chlorine solution disclosed in present invention to the respiratory system so as to treat or prevent one or more respiratory conditions.

For the applications present invention, any suitable device may be used to disperse chlorine solution into, but not limited to, spray bottle, aerosol, humidifiers, misters, foggers, vaporizers, atomizers, water sprays, and other spray devices.

The present invention is further described by reference to the following examples, which are illustrative only and not limiting of the claimed invention.

### EXAMPLE

### Following Example 1 to 4 Described the Process of Preparing Pharmaceutical Composition (LifeViroTreat)

### Example-1: Preparation of Respule

The chlorine gas (1 gm) is dissolved in the WFI grade water (1000 ml) in presence of stabilizers to form a chlorine gas solution at 0-10° C. Then chlorine gas solution is diluted into respule to make 2 mg dosage form. The respule is added into nebulizer.

### Example-2: Preparation of Respule

The chlorine gas (0.5 gm) is dissolved in the WFI grade water (1000 ml) in presence of stabilizers to form a chlorine gas solution at 0-10° C. Then chlorine gas solution is added into respule to make 5 mg dosage form. The respule is added into nebulizer.

### Example-3: Preparation of Respule

The chlorine gas (1.5 gm) is dissolved in the WFI grade water (1000 ml) in presence of stabilizers to form a chlorine gas solution at 0-10° C. Then chlorine gas solution is added into respule to make 8 mg dosage form. The respule is added into nebulizer.

### Example-4: Preparation of Vials

Aqueous chlorine solution prepared by simple solubilization method. An amount of 1000 mg Chlorine dissolved in 1000 mL of triple distilled water along with stabilizer and stirred for 15 minutes to get a uniform solution. Aqueous Chlorine solution is sterilized by filtration method and passed through 0.22μ filter. Sterilized aqueous Chlorine solution further divided in to unit 10 mL each which consist 100 ppm concentration of chloride. Furthermore, these 10 ml sterilized aqueous Chlorine solution filled in pre-sterilized vials and aseptically sealed with closure (FIG. 6 A). Filled vials further packed in primary followed by secondary carton and stored at USP cool temperature.

### Example 5: Safety and Toxicological Studies

Acute toxicological effects of Lifevirotreat composition were performed by administering a multiple inhalation doses to rodent rats and non-rodents rabbits (Table 2), followed by observation period of 14 days 3 female animals per dose level were used in this study as described in the OECD guidelines for testing of chemicals. Animals were kept in animal restrainer and connected with nebulizer. Nebulizer chamber was filled before starting the experiment with aqueous chlorine formulation (1 mg/m3) for 15 minutes (LifeViroTreat, 10 ml glass vial). Animals were exposed to the aqueous chlorine formulation for 15 minutes and for 6 and 12 times in a day. Animals were assessed for their mortality, morbidity.

TABLE 2
Experimental details for preforming safety and toxicological
studies for LifeViroTreat Composition.

| | | | No. of | | Route of |
|---|---|---|---|---|---|
| Animal Model | Dose | Frequency of dose | Animals | Sex | administration |
| Rat | Air control | — | 3 | Female | Inhalation |
| | 5 Times | LD2 | 3 | | through |
| | (4.18 mg/kg) | (Every 4 hour for 3 days) | | | Nebulizer |
| | | LD4 | 3 | | |
| | | (Every 2 hour for 3 days) | | | |
| | 10 Times | HD2 | 3 | | |
| | (8.37 mg/kg) | (Every four hour for 5 days) | | | |
| | | HD4 | 3 | | |
| | | (Every 2 hour for 3 days) | | | |
| Rabbit | Air Control | — | 3 | Female | Inhalation |
| | 5 Times | LD2 | 3 | | through |
| | (2.09 mg/kg) | (Every 4 hour for 3 days) | | | Nebulizer |
| | | LD4 | 3 | | |
| | | (Every 2 hour for 3 days) | | | |
| | 10 Times | HD2 | 3 | | |
| | (4.19 mg/kg) | Every four hour for 5 days | | | |
| | | HD4 | 3 | | |
| | | (Every four hour for 5 days) | | | |

Moreover, after the exposure period on 14 day biochemical parameters (Table 3 & table 4) and hematological (table 5 & Table 6) were investigated. Animals were evaluated for anxiety, motor activity and other behavioral changes.

TABLE 3
Effects of LifeViroTreat composition by inhalation route on some
biochemical parameters in the acute toxicity study in rats

| | Treatment Groups | | | | | |
|---|---|---|---|---|---|---|
| | | 5 Times | | 10 Times | | |
| | | (4.18 mg/kg) | | (8.37 mg/kg) | | |
| | | LD2 | LD4 | HD2 | HD4 | |
| | Control Group | (Every 4 hour | (Every 2 hour | (Every 4 hour | (Every 2 hour | Reference |
| PARAMETERS | Air Control | for 3 days) | for 3 days) | for 3 days) | for 3 days) | Values |
| ALB (g/dL) | 3.82 ± 0.27 | 3.93 ± 0.21 | 3.81 ± 0.31 | 3.67 ± 0.19 | 3.42 ± 0.44 | 3.4-4.8 |
| ALP (U/L) | 217.33 ± 18.15 | 230.00 ± 50.74 | 209.33 ± 14.72 | 178.33 ± 33.61 | 162.67 ± 7.02 | 62-237 |
| ALT (U/L) | 54.86 ± 7.29 | 45.90 ± 12.64 | 52.80 ± 4.00 | 53.50 ± 4.93 | 51.00 ± 1.25 | 18-76 |
| AST (U/L) | 95.63 ± 12.47 | 100.07 ± 15.59 | 125.47 ± 20.10 | 116.67 ± 12.15 | 102.97 ± 17.39 | 74-143 |
| BIL (mg/dL) | 0.10 ± 0.00 | 0.10 ± 0.00 | 0.10 ± 0.00 | 0.10 ± 0.00 | 0.10 ± 0.00 | 0.05-0.15 |
| CA (mg/dL) | 10.38 ± 0.61 | 10.93 ± 0.67 | 10.00 ± 1.75 | 9.23 ± 1.10 | 9.17 ± 2.11 | 9.5-11.5 |
| CHO (mg/dL) | 51.10 ± 6.27 | 59.77 ± 2.67 | 55.23 ± 6.82 | 44.83 ± 5.14 | 50.87 ± 4.35 | 37-85 |
| CREJ (mg/dL) | 0.36 ± 0.05 | 0.32 ± 0.02 | 0.30 ± 0.01 | 0.32 ± 0.07 | 0.31 ± 0.02 | 0.2-0.5 |
| PHOS (mg/dL) | 6.54 ± 1.40 | 7.57 ± 1.58 | 7.07 ± 1.72 | 7.03 ± 1.59 | 8.33 ± 1.56 | 5.58-10.41 |
| TP (g/dL) | 6.30 ± 0.54 | 6.64 ± 0.84 | 7.04 ± 0.29 | 6.81 ± 0.45 | 6.67 ± 0.06 | 5.2-7.1 |
| UREA (mg/dL) | 15.60 ± 1.62 | 17.97 ± 3.47 | 20.40 ± 1.21 | 19.10 ± 4.20 | 22.17 ± 2.35 | 12.3-24.6 |
| GLU (mg/dL) | 144.33 ± 15.28 | 133.00 ± 10.58 | 132.33 ± 14.64 | 120.00 ± 12.77 | 142.33 ± 17.24 | 70-208 |

Note:
Each value represents the mean ± standard deviation (n = 3)

TABLE 4
Effects of LifeViroTreat composition by inhalation route on some
biochemical parameters in the acute toxicity study in rabbits

| | Treatment Groups | | | | | |
|---|---|---|---|---|---|---|
| | | 5 Times | | 10 Times | | |
| | | (4.18 mg/kg) | | (8.37 mg/kg) | | |
| | | Group I | Group II | Group III | Group IV | |
| | Control Group | (Every 4 hour | (Every 2 hour | (Every 4 hour | (Every 2 hour | Reference |
| | Only Air | for 3 days) | for 3 days) | for 3 days) | for 3 days) | Values |
| ALB (g/dL) | 34.03 ± 6.09 | 3.93 ± 0.05 | 3.81 ± 0.31 | 3.67 ± 0.19 | 3.42 ± 0.44 | 23-49 |
| ALP (U/L) | 204.44 ± 17.64 | 196.67 ± 102.02 | 259.33 ± 32.52 | 278.33 ± 33.61 | 229.33 ± 51.47 | 44-402 |
| ALT (U/L) | 56.15 ± 7.29 | 45.90 ± 12.64 | 52.80 ± 4.00 | 53.50 ± 4.93 | 51.00 ± 1.25 | 34-146 |
| AST (U/L) | 22.36 ± 4.63 | 138.40 ± 18.40 * | 107.37 ± 5.41 | 106.70 ± 1.23 | 103.97 ± 2.10 | 7.5-39.7 |
| BIL (mg/dL) | 0.10 ± 0.00 | 0.10 ± 0.00 | 0.10 ± 0.00 | 0.10 ± 0.00 | 0.10 ± 0.00 | 0.019-0.2 |
| CA (mg/dL) | 10.90 ± 1.99 | 5.93 ± 0.67 | 5.00 ± 0.26 | 5.57 ± 0.45 | 5.00 ± 0.17 | 9.22-13.63 |
| CHO (mg/dL) | 53.46 ± 5.04 | 59.77 ± 2.67 | 55.23 ± 6.82 | 44.83 ± 5.14 | 50.87 ± 4.35 | 30-65 |
| CREJ (mg/dL) | 0.81 ± 0.22 | 0.32 ± 0.02 | 0.30 ± 0.01 | 0.32 ± 0.07 | 0.31 ± 0.02 | 0.5-2.6 |
| PHOS (mg/dL) | 5.12 ± 0.13 | 6.23 ± 0.06 | 5.03 ± 0.40 | 5.37 ± 0.49 | 4.67 ± 0.49 | 4-6 |
| TP (g/dL) | 60.62 + 8.64 | 7.09 ± 0.21 | 7.04 ± 0.29 | 6.81 ± 0.45 | 6.67 ± 0.06 | 49-79 |
| UREA (mg/dL) | 48.04 ± 9.41 | 34.63 ± 3.61 | 34.07 ± 4.70 | 33.17 ± 2.37 | 33.50 ± 0.75 | 25.48-71.41 |
| GLU (mg/dL) | 150.67 ± 11.06 | 136.67 ± 9.28 | 143.33 ± 13.01 # | 132.33 ± 11.59 | 140.00 ± 11.00 | 89-150 |

Note:
Each value represents the mean ± standard deviation (n = 3)
ALB2: Albumin,
ALP: Alkaline phosphatase,
ALT: Alanine Aminotransferase,
AST: Aspartate Aminotransferase,
BILT2: Total Bilirubin,
CA2: Calcium,
CHOL: Cholesterol,
CREJ: Creatinine,
PHOS: Phosphorous,
TP: Total protein,
UREL: Urea level,
GLU: Glucose

TABLE 5
Effects of LifeViroTreat composition by inhalation route on some
hematological markers in the acute toxicity study in rats

| | Treatment Groups | | | | | |
|---|---|---|---|---|---|---|
| | | 5 Times | | 10 Times | | |
| | | (4.18 mg/kg) | | (8.37 mg/kg) | | |
| | | LD2 | LD4 | HD2 | HD4 | |
| | Control Group | (Every 4 hour | (Every 2 hour | (Every 4 hour | (Every 2 hour | Reference |
| PARAMETERS | Only Air | for 3 days) | for 3 days) | for 3 days) | for 3 days) | Values |
| WBC (×10E03 | 6.62 ± 1.45 | 5.24 ± 1.26 | 7.30 ± 1.15 | 6.99 ± 1.71 | 6.14 ± 2.07 | 1.13-7.49 |
| cells/μL) | | | | | | |
| RBC (×10E06 | 7.72 ± 1.12 | 8.28 ± 0.40 | 7.63 ± 0.49 | 7.56 ± 0.18 | 7.15 ± 1.24 | 7.07-9.03 |
| cells/μL) | | | | | | |
| HGB (g/dL) | 14.59 ± 1.08 | 15.13 ± 1.31 | 13.13 ± 1.44 | 14.23 ± 2.16 | 16.00 ± 1.71 | 13.7-16.8 |
| HCT (%) | 42.00 ± 3.61 | 42.17 ± 3.70 | 39.03 ± 0.75 | 36.07 ± 0.05 | 37.90 ± 2.62 | 35-52 |
| MCV (fL) | 55.89 ± 4.06 | 50.83 ± 2.23 | 50.43 ± 2.50 | 54.33 ± 4.71 | 54.67 ± 5.11 | 49.90-60.1 |
| MCH (pg) | 18.83 ± 3.55 | 18.40 ± 2.16 | 18.49 ± 1.22 | 17.90 ± 2.15 | 17.80 ± 1.37 | 17.8-20.9 |
| MCHC (g/dL) | 35.78 ± 1.77 | 34.73 ± 6.79 | 34.47 ± 6.91 | 35.20 ± 6.63 | 35.13 ± 5.37 | 32.7-37.9 |
| RDW (%) | 12.60 ± 0.64 | 13.43 ± 1.11 | 13.70 ± 1.15 | 13.43 ± 1.96 | 14.30 ± 1.50 | 10.5-14.9 |
| PLT(×10E03 | 781.33 ± 105.64 | 710.33 ± 86.59 | 871.00 ± 24.98 | 891.67 ± 59.53 | 796.33 ± 79.43 | 680-1200 |
| cells/μL) | | | | | | |
| MPV (fL) | 8.01 ± 1.07 | 9.57 ± 1.17 | 9.37 ± 0.61 | 8.40 ± 2.13 | 9.57 ± 1.46 | 6.2-9.8 |
| % NEUT (%) | 19.27 ± 4.33 | 13.87 ± 4.78 | 10.67 ± 0.64 | 12.67 ± 4.69 | 15.03 ± 3.51 | 7.1-33.2 |
| % LYM (%) | 77.00 ± 11.19 | 78.30 ± 3.32 | 86.07 ± 2.79 | 81.87 ± 5.42 | 79.60 ± 6.91 | 62.2-90 |
| % MONO (%) | 2.69 ± 0.51 | 4.47 ± 0.15 | 2.90 ± 0.72 | 3.07 ± 0.21 | 4.77 ± 0.21 | 0.8-5.9 |
| % EOS (%) | 2.42 ± 0.52 | 1.50 ± 0.36 | 1.57 ± 0.29 | 0.80 ± 0.10 | 1.57 ± 1.24 | 0.4-4.5 |
| % BASO (%) | 0.16 ± 0.06 | 0.43 ± 0.06 | 0.60 ± 0.35 | 0.45 ± 00.02 | 0.43 ± 0.15 | 0-0.8 |

Note:
Each value represents the mean ± standard deviation (n = 3)

TABLE 6
Effects of LifeViroTreat composition by inhalation route on some
hematological markers in the acute toxicity study in rabbits

| | Treatment Groups | | | | | |
|---|---|---|---|---|---|---|
| | | 5 Times | | 10 Times | | |
| | | (4.18 mg/kg) | | (8.37 mg/kg) | | |
| | | LD2 | LD4 | HD2 | HD4 | |
| | Control Group | (Every 4 hour | (Every 2 hour | (Every 4 hour | (Every 2 hour | Reference |
| PARAMETERS | Only Air | for 3 days) | for 3 days) | for 3 days) | for 3 days) | Values |
| WBC (×10E03 | 11.64 ± 1.16 | 11.13 ± 0.80 | 9.87 ± 0.61 | 11.12 ± 1.39 | 11.33 ± 0.88 | 5.2-16.5 |
| cells/μL) | | | | | | |
| RBC (×10E06 | 5.59 ± 0.88 | 5.18 ± 0.54 | 5.79 ± 0.69 | 5.03 ± 0.33 | 4.93 ± 0.21 | 3.7-7.5 |
| cells/μL) | | | | | | |
| HGB (g/dL) | 8.56 ± 0.79 | 8.47 ± 1.27 | 6.07 ± 0.57 | 8.10 ± 1.65 | 8.7 ± 0.15 | 7.8-15.4 |
| HCT (%) | 30.14 ± 1.98 | 28.03 ± 2.81 | 29.37 ± 0.87 | 27.30 ± 1.78 | 26.73± | 26.7-47.2 |
| MCV (fL) | 53.60 ± 5.50 | 54.13 ± 0.25 | 53.57 ± 1.01 | 54.30 ± 0.00 | 57.60± | 55.0-79.6 |
| MCH (pg) | 24.93 ± 4.41 | 22.50 ± 2.40 | 24.17 ± 4092 | 23.53 ± 4.26 | 23.87± | 19.2-29.5 |
| MCHC (g/dL) | 27.39 ± 3.51 | 28.77 ± 4.58 | 25.13 ± 4.03 | 25.77 ± 3.20 | 26.50± | 25.52-37 |
| RDW (%) | 13.78 ± 1.25 | 15.80 ± 3.46 | 14.47 ± 4.57 | 15.27 ± 0.70 | 13.10± | 11.5-16.2 |
| PLT (×10E03 | 683.00 ± 95.77 | 609.33 ± 59.68 | 723.33 ± 87.51 | 764.67 ± 68.42 | 746.67 ± 83.27 | 112-795 |
| cells/μL) | | | | | | |
| MPV (fL) | 6.60 ± 0.62 | 6.13 ± 0.95 | 7.37 ± 0.38 | 6.47 ± 1.36 | 6.83± | 5.2-9.9 |
| % NEUT (%) | 50.16 ± 8.12 | 46.63 ± 1.00 | 49.40 ± 1.32 | 53.20 ± 4.17 | 50.13± | 21-73 |
| % LYM (%) | 45.88 ± 5.45 | 42.40 ± 3.87 | 39.03 ± 0.72 | 44.93 ± 4.57 | 40.53± | 9-64 |
| % MONO (%) | 9.19 ± 1.59 | 9.57 ± 1.50 | 8.73 ± 1.33 | 7.37 ± 1.90 | 2.60± | 1-15 |
| % EOS (%) | 0.45 ± 0.25 | 0.57 ± 0.23 | 0.47 ± 0.29 | 0.27 ± 0.25 | 0.23± | 0.0-0.7 |
| % BASO (%) | 8.27 ± 0.55 | 6.73 ± 2.40 | 6.33 ± 2.52 | 4.33 ± 1.55 | 6.33± | 0.5-9 |

Note:
Each value represents the mean ± standard deviation (n = 3)
WBC: White blood cells,
LY: Lymphocyte,
MON: Monocytes,
GRAN: Granulocytes,
HGB: Hemoglobin,
HCT: Hematocrit,
MCV: Mean corpuscular volume,
MCH: Mean corpuscular hemoglobin,
MCHC: Mean corpuscular hemoglobin concentration,
RDW: Red blood cell distribution width,
PLT: Platelets,
MPV: Mean platelet volume,

Result:

No mortality and morbidity were observed in any of the animals administered with the test item (Table 7). All the values were in normal range as per reference values and no significant variation observed in any parameter in all treated group as compared to normal control (Air control) Group. In all the steps, animals were in somnolence condition with decreased motor activity. None of the animals exhibit gross lesions related to test item administration. Based on the results obtained, it is concluded that, LifeViroTreat at 5 Times (4.18 mg/kg) and 10 Times (8.37 mg/kg) in rat and 5 Times (2.09 mg/kg), 10 Times (4.19 mg/kg) in rabbit was found to be safe.

TABLE 7
Summary of Mortality & Morbidity; Clinical signs and Gross pathology

| | Animal | | Frequency | Animal | | Mortality & | Day of | Gross |
|---|---|---|---|---|---|---|---|---|
| Day | Model | Dose conc | of dose | No. | Clinical Signs | Morbidity | Necropsy | Pathology |
| 1^{st} | Rat | Air | — | 1 | NAD | Nil | 14^{th} Day | NAD |
| Day | | Control | | 2 | | | | |
| | | | | 3 | | | | |
| | | 5 Times | LD2 | 1 | NAD | Nil | 14^{th} Day | NAD |
| | | (4.18 mg/kg) | (Every 4 | 2 | | | | |
| | | | hour for 3 | 3 | | | | |
| | | | days) | | | | | |
| | | | LD4 | 1 | NAD | Nil | 14^{th} Day | NAD |
| | | | Every 2 | 2 | | | | |
| | | | hour for 3 | 3 | | | | |
| | | | days | | | | | |
| | | 10 Times | HD2 | 1 | All animals were in | Nil | 14^{th} Day | NAD |
| | | (8.37 mg/kg) | Every four | 2 | somnolence condition | | | |
| | | | hour for 5 | 3 | with decreased motor | | | |
| | | | days | | activity following test | | | |
| | | | HD4 | | item administration on | | | |
| | | | (Every 2 | | day 0. | | | |
| | | | hour for 3 | | | | | |
| | | | days) | | | | | |
| 1^{st} | Rabbit | Air | — | 1 | NAD | Nil | 14^{th} Day | NAD |
| Day | | Control | | 2 | | | | |
| | | | | 3 | | | | |
| | | 5 Times | LD2 | 1 | NAD | Nil | 14^{th} Day | NAD |
| | | (2.09 mg/kg) | Every 4 | 2 | | | | |
| | | | hour for 3 | 3 | | | | |
| | | | days | | | | | |
| | | | LD4 | 1 | NAD | Nil | 14^{th} Day | NAD |
| | | | Every 2 | 2 | | | | |
| | | | hour for 3 | 3 | | | | |
| | | | days | | | | | |
| | | 10 Times | HD2 | 1 | All animals were in | Nil | 14^{th} Day | NAD |
| | | (4.19 mg/kg) | Every four | 2 | somnolence condition | | | |
| | | | hour for 5 | 3 | with decreased motor | | | |
| | | | days | | activity following test | | | |
| | | | HD4 | | item administration on | | | |
| | | | Every four | | day 0. | | | |
| | | | hour for 5 | | | | | |
| | | | days | | | | | |

NAD—No Abnormality Detected;

### TECHNICAL ADVANCEMENTS

Pharmaceutical composition of chlorine disclosed in the present invention:
Satisfy the existing needs, as well as others, overcomes the deficiencies found in the prior art.
Inhalable aqueous chlorine solution
Easy to prepare as chlorine gas is inexpensive, easy available and transportable which makes the invention robust,
is economical;
concentration of virus can be minimized with this method treatment; and
very easy way to treat the patient having respiratory infection by inhalation.

1-20. (canceled)

21. A pharmaceutical composition administered by inhalation in an individual; wherein the concentration of chlorine is in the range of 0.1 to 2 ppm on the basis of breathing air for 15 min.

22. The pharmaceutical composition claimed in claim 21, wherein inhalation time is 15 min at interval of 4 hrs for 3 days.

23. The pharmaceutical composition claimed in claim 21, wherein the concentration of chlorine by inhalation is in the range of 0.1 to 1 ppm on the basis of breathing air for 15 min at interval of 2 hrs for 6 days.

24. The pharmaceutical composition claimed in claim 21, wherein the chlorine inhalation by Individual is in the range of 0.1 to 2 ppm on the basis of breathing air for 5 min at interval of 2 hrs for 6 days.

25. The pharmaceutical composition claimed in claim 21, wherein the chlorine inhalation by Individual is in the range of 0.1 to 2 ppm on the basis of breathing air for 10 min at interval of 6 hrs for 6 days.

26. The pharmaceutical composition claimed in claim 21, wherein the chlorine inhalation by Individual is in the range of 0.1 to 2 ppm on the basis of breathing air for 10 min at interval of 12 hrs for 12 days.

27. The pharmaceutical composition claimed in any claim of 21, wherein composition administered by inhalation from 1 to 10 times per day.

28. A pharmaceutical composition claimed in claim 21, comprising an effective amount of chlorine dissolved in an inactive ingredient and/or a stabilizing agent.

29. The pharmaceutical composition claimed in claim 28, wherein the chlorine content is in the range of 1 to 200 ppm.

30. The pharmaceutical composition claimed in claim 28, wherein the chlorine content is in the range of 1 to 99 ppm.

31. The pharmaceutical composition claimed in claim 28, wherein said chlorine is chlorine gas or chlorine releasing compound.

32. The pharmaceutical composition claimed in claim 31, wherein chlorine releasing compounds are selected from mixture of calcium hypochlorite with the salt of sodium, potassium, ammonia; wherein salt of sodium, potassium, ammonia are sodium sulphate, potassium sulphate and ammonium sulphate.

33. The pharmaceutical composition claimed in claim 28, wherein said inactive ingredient is selected from distilled water, saturated edible oil, saturated neuzoil oil, saline water, glycols, higher alcohol and mixture thereof.

34. The pharmaceutical composition claimed in claim 28, wherein said inactive ingredient is distilled water.

35. The pharmaceutical composition claimed in claim 28, wherein said stabilizing agents is selected from alkali salt of mineral acids, saline water, sodium sulphate, sodium carbonate, sodium bicarbonate, potassium sulphate, potassium chloride, boric acid and mixture thereof.

36. The pharmaceutical composition as claimed in claim 28, wherein concentration of said stabilizing agent is in the range of 0.1 to 10%.

37. The pharmaceutical composition as claimed in claim 28, wherein composition is packed in amber color glass vial.

38. The pharmaceutical composition as claimed in claim 28, wherein pH of solution is in the range of 1 to 8.

39. The pharmaceutical composition as claimed in claim 28, wherein composition is administered as liquid, mist, spray, aerosol or steam.

40. A method of treating a respiratory viral infection by administrating to the subject in need thereof the pharmaceutical composition according to any of the claim 21.
